(19) United States
(12) Patent Application Publication
(10) Pub. No.: US 2014/0221747 A1
(43) Pub. Date: Aug. 7, 2014

(54) APPARATUS, SYSTEMS AND METHODS WHICH INCLUDE AND/OR UTILIZE FLEXIBLE FORWARD SCANNING CATHETER

(71) Applicants: Guillermo J. Tearney, Cambridge, MA (US); William C. Warger, II, Cambridge, MA (US); Robert Carruth, Arlington, MA (US); Lara Wurster, Boston, MA (US); Michalina Gora, Cambridge, MA (US)

(72) Inventors: Guillermo J. Tearney, Cambridge, MA (US); William C. Warger, II, Cambridge, MA (US); Robert Carruth, Arlington, MA (US); Lara Wurster, Boston, MA (US); Michalina Gora, Cambridge, MA (US)

(73) Assignee: The General Hospital Corporation, Boston, MA (US)

(21) Appl. No.: 14/170,833

(22) Filed: Feb. 3, 2014

Related U.S. Application Data
(60) Provisional application No. 61/759,859, filed on Feb. 1, 2013.
(60) Provisional application No. 61/799,272, filed on Mar. 15, 2013.

(51) Int. Cl. A61B 1/00; A61B 1/04; A61B 1/07

(52) U.S. Cl. CPC A61B 1/00172; A61B 1/07; A61B 1/04
USPC 600/111; 600/178; 600/182; 600/162; 600/109

(57) ABSTRACT

These and other objects of the present disclosure can be achieved by provision of an apparatus for illuminating a structure(s), which can include a first arrangement and a second arrangement which can each be configured to rotate and deflect a radiation(s) transmitted therethrough at an angle with respect to an axis of rotation thereof. There can be a plurality of rotating third arrangements, where at least one can be connected to the first arrangement, and at least another one can be connected to the second arrangement. A fourth arrangement can be connected to the third arrangements, and can he configured to rotate the third arrangements. One of the rotating third arrangements can be flexible, can have a length that is greater than ten times a diameter of the first arrangement or the second arrangement, can he surrounded by a housing, and/or can contain an optical waveguide arrangement extending therethrough.

### CROSS-REFERENCE TO RELATED APPLICATION(S)

This application relates to and claims priority from U.S. Patent Application Ser. No. 61/759,859 filed on Feb. 1, 2013, and U.S. Patent Application Ser. No. 61/799,272 filed on Mar. 15, 2013, the entire disclosures of which are incorporated herein by reference.

### FIELD OF THE DISCLOSURE

The present disclosure relates to exemplary embodiments of apparatus, systems and methods which can include and/or utilize flexible forward scanning catheter.

### BACKGROUND INFORMATION

Point-scanning imaging techniques require the source point to be translated (scanned) throughout a region to create an image. In a forward-scanning configuration, scanning is typically achieved with a reflective geometry to create a uniform raster scan upon the sample. However, a reflective geometry results in extra width and bulk for the device by folding the source path, thereby limiting the minimum size of the imaging device. Alternative miniature forward-scanning configurations have been developed such as resonating fiber and a tuning fork cantilever, but these techniques require a relatively long rigid length to achieve the necessary beam deviation for a useful field of view.

Accordingly, there may be a need to address and/or overcome at least some of the above-described issues and/or deficiencies.

### SUMMARY OF EXEMPLARY EMBODIMENTS

To that end, exemplary embodiments of apparatus, systems and methods which include and/of utilize flexible forward scanning catheter according to the present disclosure can be provided.

According to a particular exemplary embodiment of the present disclosure, techniques, systems and apparatus can be provided that can utilize ardor provide a flexible forward-scanning configuration with minimum rigid volume at the distal tip. In one exemplary embodiment, the apparatus can comprises a light source, such as, e.g., a laser diode or LED, which can be transmitted through an optical fiber to a lens at the distal end. The light for another electro-magnetic radiation) can be received through the same fiber or through additional optical fibers within the device, and transmitted to a detector. The exemplary apparatus can be configured to also direct light (or another electro-magnetic radiation) to the specimen at different wavelengths or by use of a broad-bandwidth light source. In yet another exemplary embodiment of the present disclosure, the light (or another electro-magnetic radiation) returned from the specimen can be detected by one or more point detectors, one- or two-dimensional array of detectors, CCD or CMOS camera, or the like. It is possible to utilize any of the following optical imaging technology, such as, e.g., OCT, TD-OCT, SD-OCT, OFDI, SECM or fluorescence confocal microscopy and video imaging. It should be understood that other imaging technologies can be utilized in accordance with the exemplary embodiments of the present disclosure.

Further features and advantages of the exemplary embodiment of the present disclosure will become apparent taken in conjunction with the accompanying Figs. and drawings and upon reading the following detailed description of the exemplary embodiments of the present disclosure.

These and other objects of the present disclosure can be achieved by provision of an apparatus for illuminating a structure(s), which can include a first arrangement and a second arrangement winch can each be configured to rotate and deflect a radiation(s) transmitted therethrough at an angle with respect to an axis of rotation thereof. There can be a plurality of rotating third arrangements, where at least one can he connected to the first arrangement, and at least another one can be connected to the second arrangement. A fourth arrangement can be connected to the third arrangements, and can be configured to rotate the third arrangements. One of the rotating third arrangements can be flexible, can have a length that is greater than ten times a diameter of the first arrangement or the second arrangement, can be surrounded by a housing, and/or can contain an optical waveguide arrangement extending therethrough.

In certain exemplary embodiments of the present disclosure, the optical waveguide arrangement can include an optical fiber. At least one of the first arrangement or the second arrangement can include a prism, a grism, a Fresnel prism, a grading or a polished ball lens. An optical waveguide fifth arrangement can be configure to receive electro-magnetic radiation from the structure(s). A sixth arrangement can have a predetermined configuration which, upon impact by or transmission of an electro-magnetic radiation, can alter a characteristic(s) of the electro-magnetic radiation. The characteristic(s) can be intensity, reflectivity or path length of the electro-magnetic radiation.

In some exemplary embodiments of the present disclosure, the fourth arrangement can include a motor. One of the third arrangements can include a drive shaft. In certain exemplary embodiments of the present disclosure, a detection arrangement can detect an electro-magnetic radiation provided from the structure(s), which can be associated with the radiation(s) forwarded to the structure by the first and second arrangements. The detection arrangement can generate information based on the detected electro-magnetic radiation, and the information provided can be data regarding a pattern(s) of illumination of the radiation(s) on the structure(s).

According, to particular exemplary embodiments of the present disclosure, an imaging arrangement can be configured to generate and correct for an image of a portion(s) of the structure based on the pattern(s) and the data. For example, at least two of the third arrangements can be coaxial, and/or the first and second arrangements can be coaxial. There can be at least three third arrangements. In some exemplary embodiments of the present disclosure, an imaging arrangement can be configured to generate a plurality of images of the portion(s) of the structure(s) using information provided by the at least three third arrangements. The imaging arrangement can cause the images to overlap so as to generate a stereo image.

In some exemplary embodiments of the present disclosure, the first and second arrangements can have a diameter less than 6 mm, and a combination of the first and second arrangements can have length less than 10 mm. The length of the third arrangement can be greater than 15 cm, and the diameter of the third arrangement can be less than 4 mm.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF DRAWINGS

Further objects, features and advantages of the present disclosure will become apparent from the following detailed description taken in conjunction with the accompanying Figs. showing illustrative embodiment of the present disclosure, in which:

FIGS. 1 and 1B are schematic diagrams of exemplary embodiments of a forward scanning device, which utilizes one or more components to bend light at a deviation angle while the components are be rotated independently;

FIGS. 2A-2C are schematic diagrams of the apparatus which producing a scan pattern in the forward direction, according to an exemplary embodiment of the present disclosure;

FIG. 3A is a schematic diagram of a forward scanning probe according to an exemplary embodiment of the present disclosure;

FIG. 3B is a set of pictures of a scanning pattern obtained from an exemplary probe according to an exemplary embodiment of the present disclosure with a HeNe laser light source compared to a corresponding image from the simulation;

FIG. 4 is a schematic diagram of two or more angle-polished ball lenses deviation devices according to an exemplary embodiment of the present disclosure;

FIG. 5 is a schematic diagram of the coaxial forward scanning probe according to another exemplary embodiment of the present disclosure;

FIG. 6 is a schematic diagram of the coaxial forward scanning probe according to still another exemplary embodiment of the present disclosure;

FIGS. 7A and 78 are exemplary illustrations of et another exemplary embodiment the device according to the present disclosure that has an external window element; and

FIGS. 8A and 8B are exemplary schematic diagrams of the coaxial forward scanning probe according to another exemplary embodiment of the present disclosure.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures and appended claims.

### DETAILED DESCRIPTION OF EXEMPLARY EMBODIMENTS

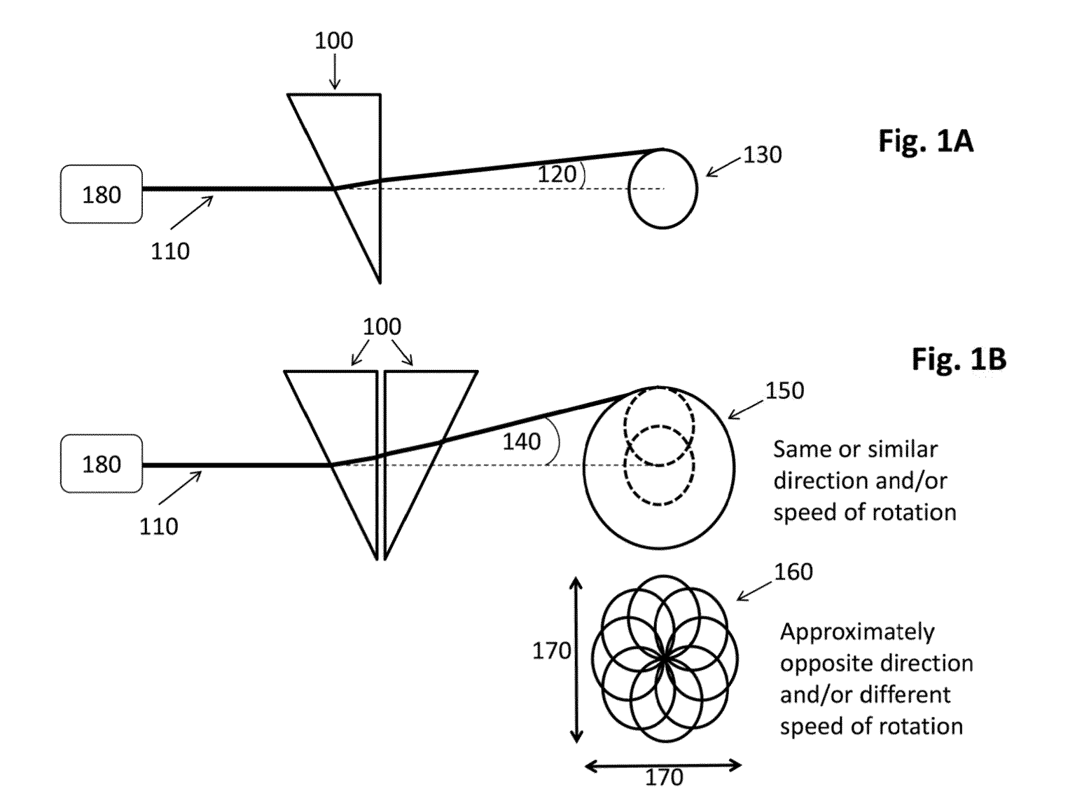

FIGS. 1A and 1B depict exemplary embodiments of a forward scanning device according to the present disclosure, which can utilizes one or more components 100 to bend the light at a deviation angle 120, 140, while the components can be rotated independently. For example, with a single deviation device 100, the light 110 (or other electromagnetic radiation) from the light source 180 (or another energy providing arrangement) after passing through the device 100 can scan a circle 130 with a diameter dependent on the deviation angle 120 and distance between the deviation device 100 and the observation point of the scan pattern (as shown in FIG. 1A).

According to the exemplary embodiment shown in FIG. 1B having two deviation devices 100, the light 110 (or other electromagnetic radiation) can be deviated at an angle 140 that is the sum of the deviations from the two devices 100. For example, if the two deviation devices 100 are rotated at the same speed and in the same direction, the light can scan a circle 150. If the two deviation devices 100 are rotated at the same speed and in opposite directions, the light can scan a line. If the two deviation devices 100 are rotated at different speeds and in the same direction, the light can scan a spiral pattern. If the deviation devices 100 are rotated at different speeds and in opposite directions, the light can scan a rosette pattern 160.

The density of the sampled region produced by the scan pattern can be at least partially dependent on the relation of the rotation speeds and the speed of the data acquisition. Depending on the rotation speeds different scanning patterns can achieved, if the prime numbers are used the scan pattern will not repeat the same scanning path. In the preferred embodiment, the deviation angle of both devices can be the same, in order to sample all points within a circular region of the field of view 170, although the exemplary deviation angles can be different to sample, e.g., a ring or donut field of view. In the exemplary embodiment shown in FIG. 1B, the deviation angles can be produced with the use of similar or identical prisms 100, angle polished GRIN lenses, gratings, dispersion-corrected refracting devices (GRISM), off-set lenses, acousto-optic devices driven at the same frequency, PZT/cantilever fibers and/or the like.

According to further exemplary embodiments of the present disclosure, a single device with the ability to change the deviation angle can be rotated such as an acousto-optic or electro-optic device.

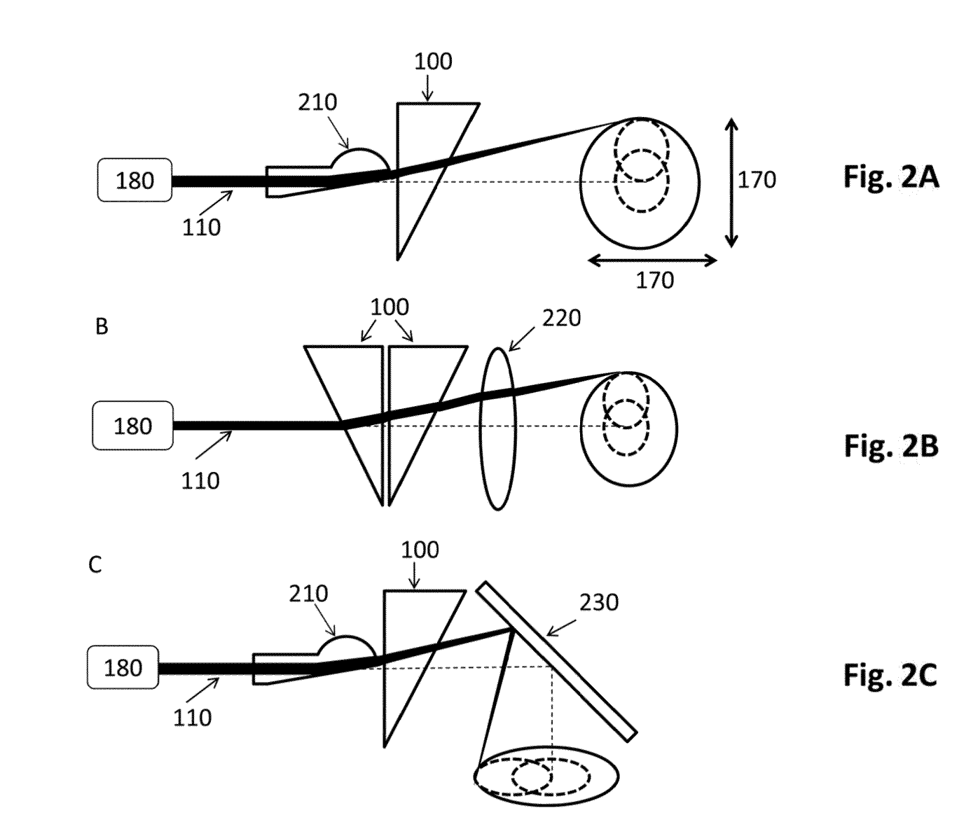

In yet another exemplary embodiment of the present disclosure that is shown in FIG. 2A, the deviation angles can be produced from the combination of different devices, such as an angle-polished ball lens 210 and the prism 100 and/or any combination of devices described herein. In this exemplary embodiment, the ball lens 210 can focus the light (or other electromagnetic radiation) within the field of view 170. In another exemplary embodiment, both of the deviation devices can focus the light or other electromagnetic radiation). According to yet another exemplary embodiment, either or both of the deviation devices can output collimated light for other electromagnetic radiation) from a light source 180 for another energy providing arrangement) that can be scanned by the deviation devices 210, 100. According to a further exemplary embodiment of the present disclosure that is shown in FIG. 2B, an additional lens 220 at the distal tip of the apparatus can focus the collimated output within the field of view 170. In another exemplary embodiment, the lens 220 can have zoom and/or translation capabilities to adjust the field of view.

FIGS. 2A and 2B depict additional exemplary embodiments of the present disclosure, in which the exemplary apparatus can produce a scan pattern in the forward direction. According to an exemplary embodiment shown in FIG. 2C, a reflective surface 230 can be positioned at the distal tip to create a side-viewing device. In yet another exemplary embodiment, a third deviation device can be included to offset the field of view at a desired angle.

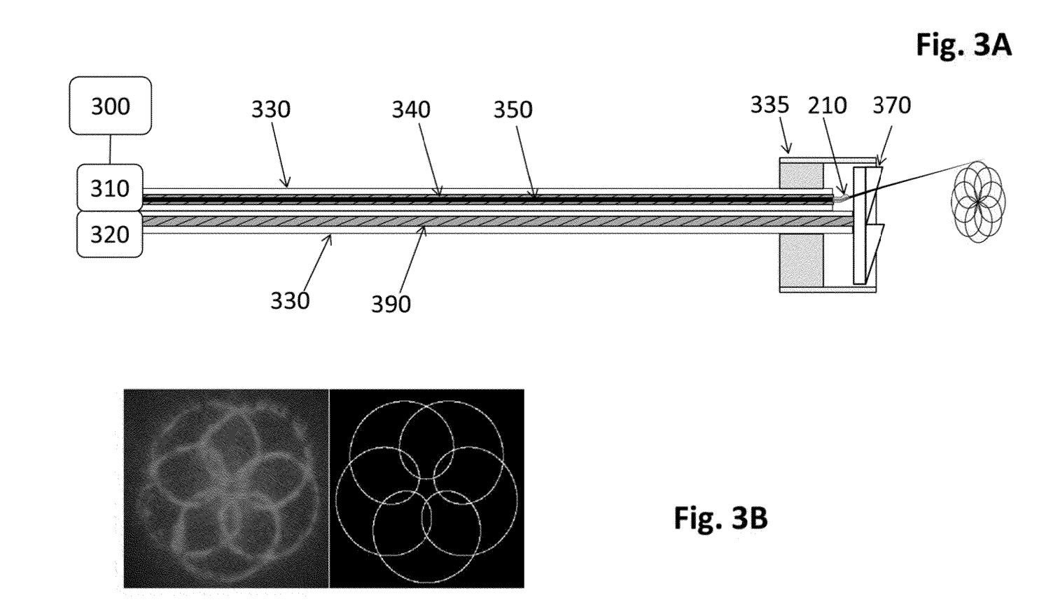

An exemplary embodiment of a forward scanning probe according to the present disclosure is illustrated in FIG. 3A. For example, a distal tip of the exemplary forward probe can have a configuration similar to the exemplary configuration shown in FIG. 2A, with the angle-polished ball lens 210 focusing and collecting the light (or other electromagnetic radiation) from and to the imaging system 300 transmitted over an optical fiber 350 and a repetitive symmetric sheet of deviation material such as a Fresnel-prism sheet 370, grating, off-set lenslet array, or the like. The exemplary deviation devices can be rotated by parallel miniature drive shafts 340, 390 that connect the deviation devices at the distal tip with motors 310, 320, air bearings, or the like at the proximal tip. In further exemplary embodiments of the present disclosure, the deviation devices can be rotated by miniature motors at the distal tip of the apparatus or can be mounted in a magnetic bearing that can be driven by an external magnetic or electric fields applied around the object being imaged.

As illustrated in FIG. 3A, e.g., a mount 335 can be provided to balance the deviation devices, which are generally not symmetric, to reduce and/or prevent wobble during the rotation. In this exemplary embodiment, drive shafts 340, 390 can be enclosed in a stationery protective sheath 330. FIG. 3B shows a picture of an exemplary scanning pattern (on a left panel) obtained from a prototype probe similar to the one illustrated on the right side of FIG. 3A with a HeNe laser light source. The right panel of the FIG. 3B illustrates a corresponding image from the simulation. The exemplary probe has a distal scanning head that comprises deviation devices which are enclosed in a mount and has diameter of, e.g., about 3.9 mm and length of, e.g., about 4 mm. The scanning head can be connected to the proximal motors using two or more spinning driveshaft enclosed in tethers with a diameter of e.g., about 1 mm each and length of e.g., about 1.6 m.

In one exemplary embodiment of the present disclosure, the deviation devices can be rotated with two or more separate motors. In another exemplary embodiment, the deviation devices can be rotated with a single motor with a differential between the two drive shafts or the like. According to yet another exemplary embodiment of the present disclosure, the deviation devices can be mounted with air bearings with a different number of fins or another mechanism to drive the bearings at different speeds with a single air input.

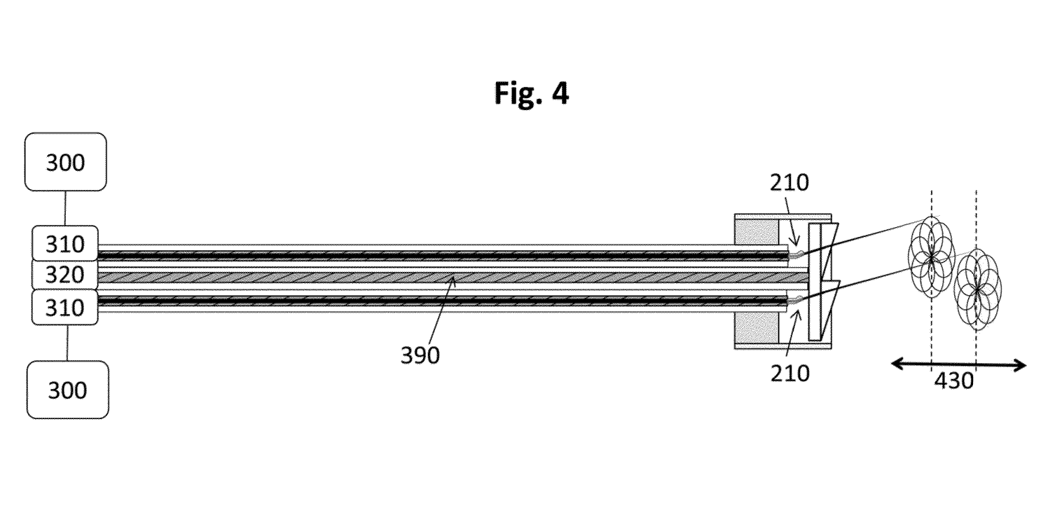

FIG. 4 shows the exemplary device (e.g., including the forward scanning probe) according to another exemplary embodiment of the present disclosure with two or more angle polished ball lenses deviation devices 210 as described at FIG. 3A. Such exemplary deviation devices 210 can be positioned next to or near the driveshaft 390 or similar spinning mechanism attached to the center of the first deviation device. In a further exemplary embodiment, an array of fibers can surround the driveshaft or similar to acquire an image front each fiber separately. According to yet another exemplary embodiment of the present disclosure, each fiber within the array can have a slightly different path length and/or focal length to create a large depth of field 430 of the final reconstructed image. In still another exemplary embodiment, the fibers can have the same path length and a mapping algorithm/procedure can be provided and/or utilized to produce a single large or densely sampled image.

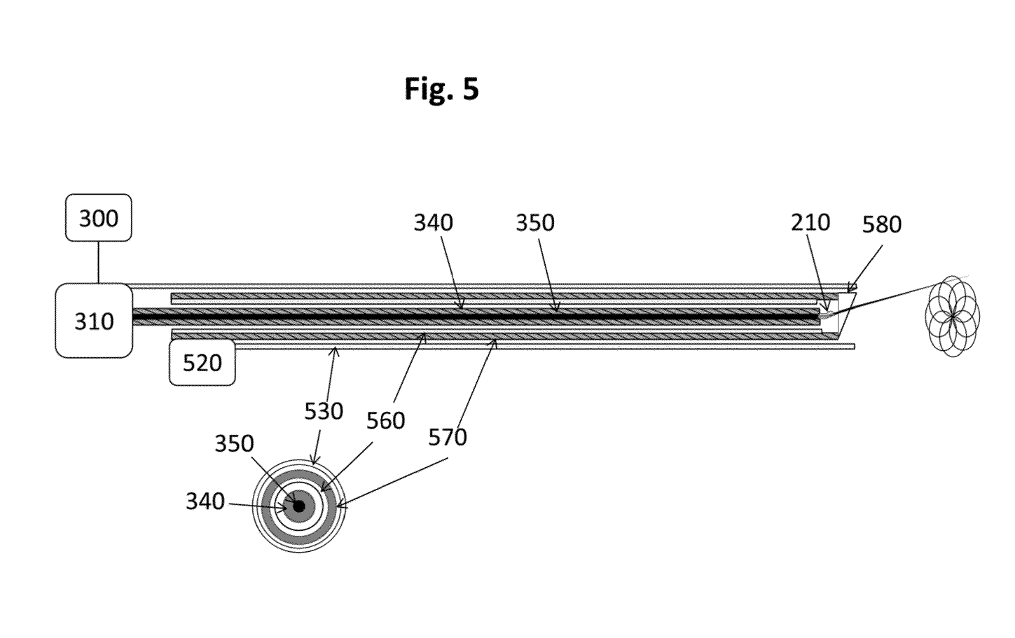

In still another exemplary embodiment of the exemplary device shown in FIG. 5, to reduce the size of the device, the one or more angle-polished ball lens deviation devices 210 can be rotated using the miniature driveshaft 340 enclosed inside of a larger driveshaft 570 rotating the second deviation device such as prism 580 in front. With such coaxial configuration of the device according to this exemplary embodiment, the outer spinning driveshaft 570 can be enclosed in a protective outer sheath 530. In another exemplary embodiment of the present disclosure, an additional sheath 560 or a Teflon layer can be added between driveshaft in order to lower friction. The outer driveshaft 570 can be rotated using off center belt motor 520 or alike.

According to yet another exemplary embodiment, miniature drive shafts, motor shafts, or the like can be attached to the center of the deviation devices. In a further exemplary embodiment, the miniature driveshaft, motor shaft, or the like can be attached to an internal gear to reduce the size of the device.

In a further exemplary embodiment of the present disclosure, encoders can be positioned on the motors to determine the rotation angle of the deviation devices. In addition, a spot, line, or the like can be placed on the deviation devices to provide a zero location within the rotation of each device that can be interpreted, within the image, by separate fibers, electrical wires, or camera within the apparatus, or by a magnet placed outside of the object being imaged. According to still another exemplary embodiment of the present disclosure, a unique pattern can be traversed by the light (or other electromagnetic radiation) that can be interpreted and reconstructed within the image.

The exemplary prisms can be attached to the shafts of two miniature motors. An optical fiber directs light through the prism to create a scan pattern on the sample. The fiber(s) in another exemplary embodiment can be associated with a miniature lens. The device can be surrounded by a sheath. In addition or alternatively, the scan pattern can be deflected in a direction that is substantially perpendicular to the axis of the probe. In yet another exemplary embodiment, the device can contain one motor and one driveshaft.

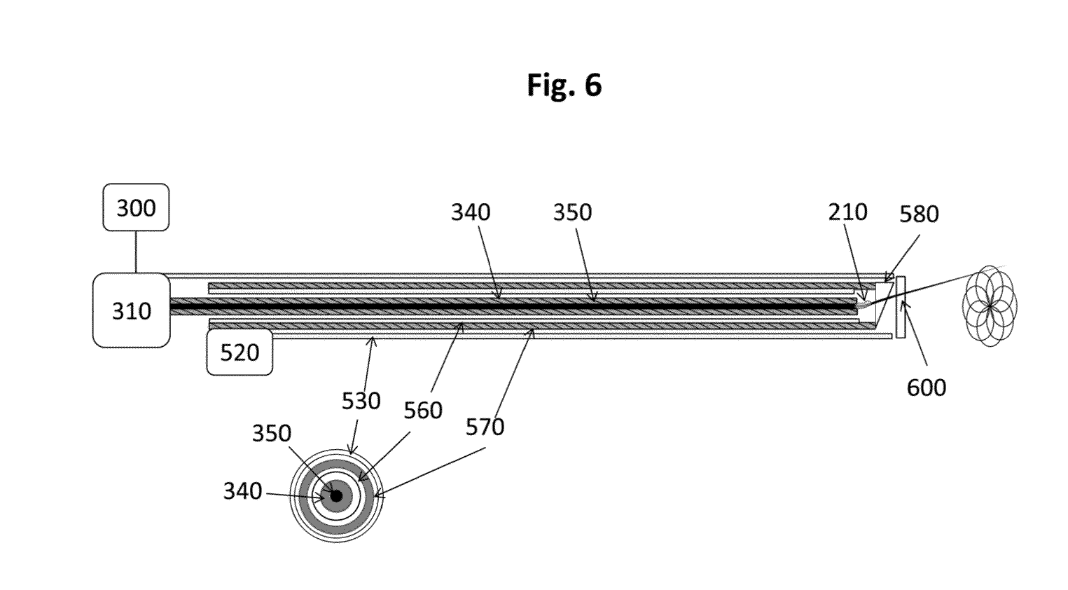

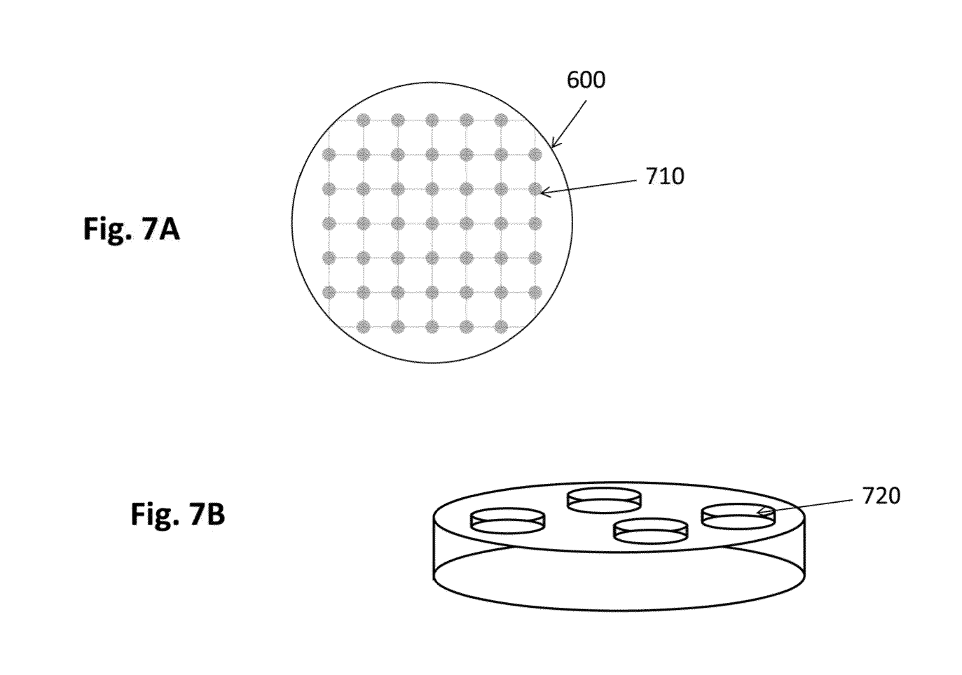

FIG. 6 illustrates the device/system according to still another exemplary embodiment of the present disclosure that has an external window element 600. The exemplary window element 600 can contain markings 710 and/or structures (see FIGS. 7A and 7B) that can be detected by the imaging system to calibrate the image and remap the spirograph scan to Cartesian coordinates. In one exemplary embodiment of the present disclosure, the markings can be or include local regions areas that absorb light or reflect light. According to a further exemplary embodiment of the present disclosure, the markings may be local regions with different refractive indices or elevations 720. In still another exemplary embodiment of the present disclosure, the imaging technology is a coherence gating technology, for example, OCT, SD-OCT, OFDI, or the like where the markings can be visualized and discriminated based on their axial position with respect to the reference arm or another structure that is seen in the image. In yet another embodiment, these markings are at known locations. A calibration image can be acquired to determine predetermined mappings for correcting the spatial coordinates of the scan pattern.

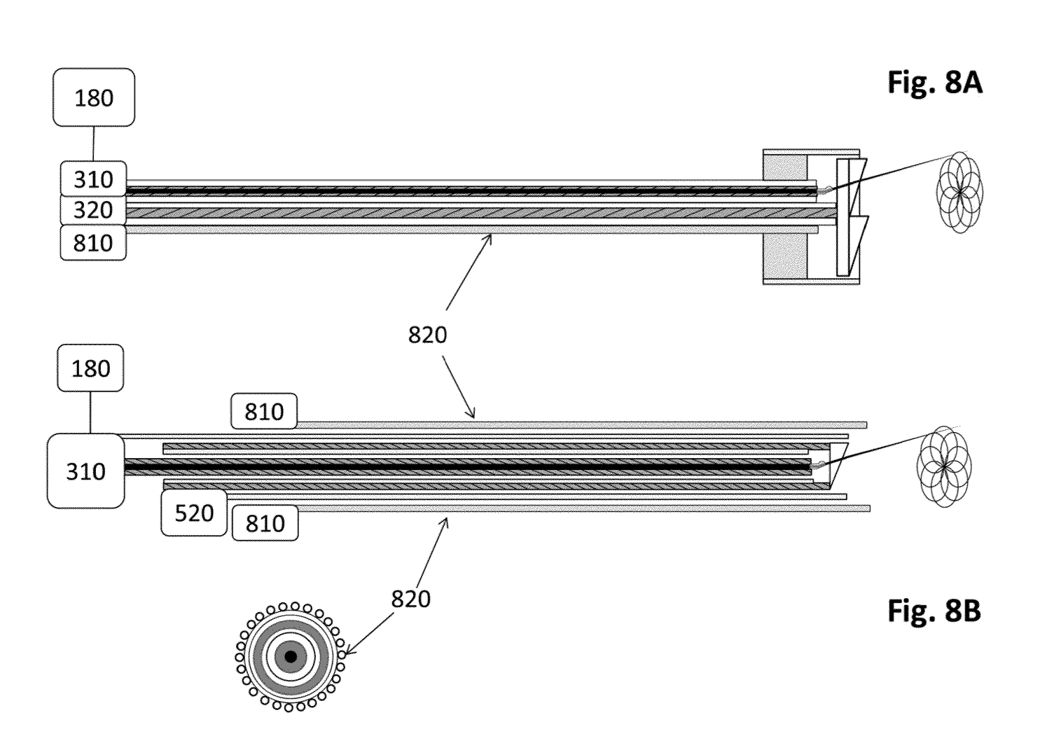

According to yet another exemplary embodiment, as shown in FIGS. 8A and 8B, additional one or more fibers 820 can be attached to the center of the exemplary probe or on its outside circumference in order to transmit light collected from the tissue to a detector 810. In further exemplary embodiments according to the present disclosure, the exemplary apparatus/systems described herein can be used to produce a scan pattern on an anatomical structure. In yet another exemplary embodiment of the present disclosure, the exemplary apparatus/system can be attached or otherwise connected to as tether, and/or may be contained or provided within a swallowable capsule. In yet a further exemplary embodiment of the present disclosure, the exemplary apparatus/system can be implanted into a biological structure.

The foregoing merely illustrates the principles of the disclosure. Various modifications and alterations to the described embodiments will be apparent to those skilled in the an in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No, 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly being incorporated herein in its entirety. All publications referenced above can be incorporated herein by reference in their entireties.

What is claimed is:

1. An apparatus for illuminating at least one structure, comprising:
a first arrangement and a second arrangement, wherein the first and second arrangements are each configured to rotate and deflect at least one radiation transmitted therethrough at an angle with respect to an axis of rotation thereof;
a plurality of rotating third arrangements, at least one of which is connected to the first arrangement, and at least another one of which is connected to the second arrangement; and
a fourth arrangement, connected to the third arrangements, and configured to rotate the third arrangements, wherein at least one of the rotating third arrangements at least one of:
(i) is flexible,
(ii) has a length that is greater than ten times a diameter of at least one of the first arrangement or the second arrangement,
(iii) is surrounded by a housing, or
(iv) contains an optical waveguide arrangement extending therethrough.

2. The apparatus according to claim 1, wherein the optical waveguide arrangement an optical fiber.

3. The apparatus according to claim 1, wherein at least one of the first arrangement or the second arrangement includes at least one of a prism, a grism, a Fresnel prism, a grading, or a polished ball lens.

4. The apparatus according to claim 1, further comprising an optical waveguide fifth arrangement which receives an electro-magnetic radiation from the at least one structure.

5. The apparatus according to claim 4, further comprising a sixth arrangement that has a predetermined configuration which, upon an impact by or a transmission of an electro-magnetic radiation, alters at least one characteristic of the electro-magnetic radiation.

6. The apparatus according to claim 5, wherein the at least one characteristic is intensity, reflectivity, or path length of the electro-magnetic radiation.

7. The apparatus according to claim 1, wherein the fourth arrangement includes a motor.

8. The apparatus according to claim 1, wherein at least one of the third arrangements includes a drive shaft.

9. The apparatus according to claim 5, further comprising a detection arrangement which detects an electro-magnetic radiation provided from the at least one structure which is associated with the at least one radiation forwarded to the structure by the first and second arrangements.

10. The apparatus according to claim 9, wherein the detection arrangement is configured to generate information based on the detected electro-magnetic radiation, and wherein the information provides data regarding at least one pattern of illumination of the at least one radiation on the structure.

11. The apparatus according to claim 10, further comprising an imaging arrangement which is configured to generate and correct for an image of at least one portion of the structure based on the at least one pattern and the data.

12. The apparatus according to claim 1, wherein at least two of the third arrangements are coaxial.

13. The apparatus according to claim 1, wherein the first and second arrangements are coaxial.

14. The apparatus according to claim 1, wherein a number of the third arrangements is at least three.

15. The apparatus according to claim 14, further comprising an imaging arrangement which is configured to generate a plurality of images of at least one portion of the structure using information provided by the at least three third arrangements.

16. The apparatus according to claim 15, wherein the imaging arrangement causes the images to overlap so as to generate a stereo image.

17. The apparatus according to paragraph 1, wherein the first and second arrangements have a diameter less than about 6 mm.

18. The apparatus according to claim 1, wherein the first and second arrangements, when combined, have a length less than about 10 mm.

19. The apparatus according to claim 1, wherein a length of the third arrangement is greater than about 15 cm.

20. The apparatus according to claim 1, wherein a diameter of the third arrangement is less than about 4 mm.
